(19) United States
(12) Patent Application Publication
(10) Pub. No.: US 2002/0010333 A1
(43) Pub. Date: Jan. 24, 2002

(54) Use of xanthine derivatives for reducing the pathological hyperreactivity of eosinophilic granulocytes, novel xanthine compounds and process for their preparation

(75) Inventors: Ulrich Gebert, Glashutten (DE); Hiristo Anagnostopulos, Wiesbaden (DE); Claus Kroegel, Merzhausen (DE); Alexander Kapp, Freiburg (DE)

Correspondence Address: FINNEGAN, HENDERSON, FARABOW, GARRETT &, DUNNER LLP, 1300 I STREET, NW, WASHINGTON, DC, 20005, US

(73) Assignee: Hoechst Aktiengesellschaft

(21) Appl. No.: 09/779,100

(22) Filed: Feb. 8, 2001

Related U.S. Application Data
(63) Continuation of application No. 09/540,405, filed on Mar. 31, 2000, now abandoned.
Application No. 09/540,405 is a continuation of application No. 08/718,340, filed on Oct. 23, 1996, now abandoned.
Application No. 08/718,340 is a 371 of international application No. PCT/US95/01060, filed on Mar. 21, 1995.

(30) Foreign Application Priority Data
Apr. 5, 1994 (DE) P 4411660.8

(51) Int. Cl. A61K 31/52; C07D 473/04

(52) U.S. Cl. 544/267; 514/263

(57) ABSTRACT

Use of xanthine derivatives for reducing the pathological hyperreactivity of eosinophilic granulocytes, novel xanthine compounds and process for their preparation.

Tertiary 1-(hydroxyalkyl)-4-alkylxanthines are suitable for the production of pharmaceuticals for the treatment of disorders which is associated with a pathologically increased reactivity of eosinophilic granulocytes. Novel xanthine derivatives and process for their preparation are described.

### DESCRIPTION

Use of xanthine derivatives for reducing the pathological hyperreactivity of eosinophilic granulocytes, novel xanthine compounds and process for their preparation.

The present invention relates to the use of tertiary 1-(hydroxyalkyl)-3-alkylxanthines for the production of pharmaceuticals for the treatment of disorders which are associated with a pathologically increased reactivity of eosinophilic granulocytes, novel xanthine compounds having the abovementioned substitution pattern and process for their preparation.

The hyperreactive eosinophilic granulocyte is the focus of attention of the pathogenesis of certain pulmonary, cardiac and cutaneous disorders which are mainly classified as of the atopic type.

The atopic type embraces disorders having an allergic diathesis, which are caused on the basis of a specifically modified level of immunity by exogenous, noninfectious substances (environmental allergens). Allergic disorders can in principle concern all main organ systems of the human body and are manifested in a multiplicity of different clinical symptoms such as arthalgias, asthma, erythema exsudativum multiforme, enteritis, nephritis, rhinitis or vasculitis (Wien Klin Wochenschr (1993) 105/23: 661-668).

In clinical practice, the immunoglobulin E (IgE)-mediated immune reactions (type I allergies) dominate in the form of anaphylaxis, allergic bronchial asthma, allergic rhinitis and conjunctivitis, allergic urticaria, allergic gastroenteritis and atopic dermatitis. There seems to be a genetic predisposition here for the readiness to react to substances from the natural environment (e.g. grass pollen, spores, house dust and mites, animal hair or food) with a hypersensitivity of the immediate type mediated by atopic antibodies (reagins). The incidence rate is currently approximately 10% of the population (Pschyrembel, Klinisches Wörterbuch [Clinical Dictionary], Walter de Gruyter-Verlag, 255th Edition, 1986, page 148) with continually rising prevalence, in particular in the industrialized countries.

It is a worrying finding that, despite intensive attempts to improve possibilities of diagnosis and therapy, the global increase in morbidity is also linked with a rise in mortality, for example in bronchial asthma (Deutsche Apotheker Zeitung (1993) 133/18: 1635-1636). Asthma—characterized by inflammatory processes with progressive, irreversible damage to the airways—is thus the only chronic disorder in the industrialized countries, in which, as a result of inadequate therapy, the number of cases of death are rising (Therapiewoche (1993) 43/7: 340-341)

According to the present state of knowledge, chronic inflammation is the focal point of the pathogenetic process, in which a multiplicity of immunocompetent cells is involved with the release of proinflammatory mediators. It is assumed here that in the acute phase of the inflammation, the so-called early-phase or immediate reaction, mainly basophilic granulocytes and mast cells are involved, whereas for the chronic symptomatology with progressive tissue death and loss of function in the late-phase reaction eosinophilic granulocytes and possibly also neutrophthlic granulocytes play the main part (Münch. med. Wschr. (1993) 135/5: 52).

Basophilic granulocytes and mast cells, also known as histaminocytes, not only release histamine, but also numerous other inflammatory mediators, after activation by binding of IgE produced in B lymphocytes to specific high-affinity receptors on the cell surface and subsequent crosslinking of the bound IgE molecules by the antigen concerned. These mediators include proteases, lipid mediators, such as the platelet activating factor (PAF), prostaglandins and leukotrienes, as well as a wide spectrum of cytokines (Immunopharmacology (1994) 27: 1-11).

In the main, these mediators have a vaso- and broncho-constrictive action, increase mucus secretion and intervene in hemostasis regulation. Moreover, chemotactic properties are ascribed to them, which make them capable of mobilizing further cells involved in the inflammation process, inter alia also the eosinophilic granulocytes responsible for the late-phase reaction which, after activation by degranulation, likewise secrete inflammatory mediators, whereby the inflammation process is perpetually maintained and its conversion to the chronic phase is initiated. The eosinophilic granulocyte is a highly potent effector cell having marked leukocyte-specific properties, such as chemotaxis, adherence, phagocytosis, release of granula proteins and formation and secretion of lipid mediators and reactive oxygen species, which make their dominant role in the pathogenetic process of the allergic inflammatory reaction intelligible (Dt. Arzteblatt (1992) 89/43, A₁: 3574-3585).

The event is initiated after allergen exposure by recruitment of eosinophils from the bone marrow and their targeted invasion into the tissue affected by the antigen, which leads to a local eosinophilia with subsequent cell activation. Various immunocompetent cells, such as T-helper cells of the Th₂ type, macrophages, neutrophils, mast cells and eosinophils themselves, which produce and secrete a number of factors responsible for differentiation, proliferation, migration and activation are involved in these pathophysiological processes.

These include the immunomodulating cytokines, such as the eosinophil-selective interleukin-5 (IL-5) formed by the T-helper cells, which controls both the differentiation and proliferation and the functional activation of the eosinophilic granulocytes, and the granulocyte/macrophage colony stimulating factor (GM-CSF) with marked cell-activating action; and also the chemotactic factors simultaneously responsible for migration and activation, such as PAF and leukotriene B₄ (LTB₄) Independently of this, the complement cleavage product C5a also has potent chemotactic and cell-stimulating activity for eosinophils.

The activated eosinophilic granulocyte for its part also reacts with mediator synthesis and release in the form of granular proteins, lipid mediators and cytotoxic oxygen metabolites.

The proinflammatory lipid mediators include, in particular, leukotriene C₄ (LTC₄), thromboxane A₂ (TXA₂) and, in turn, PAF, which increase vascular permeability, cause vasoconstriction and obstruction of the bronchi and stimulate mucus production (Pharmazie in unserer Zeit (1992) 21/2: 61-70). Among the protein mediators, eosinophil peroxidase (EPO) impress with enzymatic action and especially the nonenzymatic, basic proteins particularly relevant for the destructive processes, such as the major basic protein (MBP), the eosinophil cationic protein (ECP) and the eosinophil-derived neurotoxin/eosinophil protein X (EDN/EPX). Prominent among their varied biological properties are cytotoxic effects on a wide spectrum of cells, which extends from parasites via bronchial epithelial cells, nerve cells, cardiac muscle cells up to tumor cells. Together with the secreted reactive oxygen metabolites, they therefore contribute crucially to tissue destruction with progressive loss of function in areas of allergic inflammation reactions.

Moreover, they also stimulate histamine release from mast cells and thus induce, in the sense of a vicious circle, new early-phase attacks again.

The highly toxic protein mediators are additionally ascribed great diagnostic importance, as in patients with disorders of the atopic type, increased ECP concentrations, in particular, can be detected in the serum and in other body fluids, such as the bronchoalveolar lavage, the sputum and nasal secretions, but also deposits of these proteins in the affected tissues as a sign of the eosinophil activation which has taken place, the ECP serum level correlating significantly with the degree of severity of the disorder, so that this parameter appears suitable both for objectivizing the disease activity and for assessing the success of treatment after therapeutic intervention (Therapiewoche (1991) 41/45: 2946-2947).

The pathogenetic relationships described in the case of disorders of the atopic type make it clear that the allergic inflammation process which is the focal point with its early- and late-phase reaction is the result of a complex interaction of immune cells and their inflammatory mediators, and that therapeutic advances are only to be expected of a multifunctional pharmaceutical which both blocks the mediators of the immediate reaction and is able in a lasting manner to inhibit the recruitment and, especially, the activation of the eosinophils in the chronic late-phase reaction (Pharmazeutische Zeitung (1992) 137/5: 249-258; Agents and Actions (1991) 32/1+2: 24-33)

Surprisingly, it has now been found that 1,3-dialkylxanthines of the formula I having a tertiary hydroxyl function in the alkyl radical in position 1 fulfil the aforementioned requirements of a therapeutic suitable for the treatment of disorders of the atopic type.

1-(5-Hydroxy-5-methylhexyl)-3-methylxanthine is described in the publication WO 87/00523. It is proposed there for the treatment of peripheral and cerebral circulatory disorders and mitochondrial mypopathies, but without any information on its utility for the reduction of pathological hyperreactivity of eosinophilic granulocytes and thus for the treatment of atopic disorders being given.

Admittedly, numerous xanthine compounds are known which, on account of their phosphodiesterase-inhibitory action, have bronchospasmolytic activity and are therefore suitable for the prophylaxis and symptomatic treatment of the acute bronchospasm induced by mediators in the course of the asthmatic early reaction, but do not allow a curative therapy of atopic disorders, as they leave unaffected the underlying condition, the eosinophil-mediated, chronic inflammation process of the late-phase reaction. The most prominent representatives of this group of substances is theophylline.

More recently, a few 8-substituted 1,3-dialkylxanthines (EP 389 286; WO 92/11260), 1,3,7-trialkylxanthines (EP 421 587) and also 7-sulfonylated 1,3-dialkylxanthines and 1,3,7,8-tetrasubstituted xanthines (WO 92/11260) have also been reported which should reduce the number of eosinophils in the blood in an animal model having artificially induced eosinophilia. It was not possible, however, to show an inhibition of the functional state of the eosinophils, which is pathologically raised in atopic disorders and in the final analysis determines the course of the disease, principally in the tissue affected by the allergic inflammation process, so that the therapeutic worth of the compounds described is not confirmed. On the other hand, on the level of the cellular mediators relevant for the disease event, the compounds of the formula I show that they inhibit the early-phase reactions and, in the context of the late-phase reaction, inhibit not only the recruitment of the eosinophils, but also reduce their pathological hyperreactivity in the target tissue and thus selectively switch off the effector functions of this highly potent inflammation cell at the centre of the chronic disease process.

The publication EP 544 391 proposes the 1,3,7-trialkylated xanthines pentoxifylline (3,7-dimethyl-1-(5-oxohexyl)xanthine), propentofylline (3-methyl-1-(5-oxohexyl-7-propylxanthine) and torbafylline (7-ethoxymethyl-1-(5-hydroxy-5-methylhexyl)-3-methylxanthine) for the topical treatment of psoriasis and atopic dermatitis, but without any indication that 1.) these xanthine derivatives are also active in nontopical use or 2.) can also be employed topically or even nontopically against other disorders of the atopic type.

The invention thus relates to the use of at least one compound of the formula I

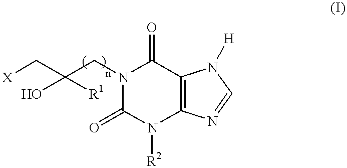

and/or a physiologically tolerable salt of the compound of the formula I and/or a stereoisomeric form of the compound of the formula I, where

R¹ is a methyl or ethyl group,

R² is an alkyl group having 1 to 4 carbon atoms and

X is a hydrogen atom or a hydroxyl group and

n is an integer from 1 to 5,

for the production of pharmaceuticals for the reduction of the pathological hyperreactivity of eosinophilic granulocytes. The compound of the formula I is particularly suitable for the prophylaxis and treatment of atopic disorders such as anaphylaxis, allergic bronchial asthma, allergic rhinitis and conjunctivitis, allergic urticaria, allergic gastroenteritis or atopic dermatitis.

Preferably, those compounds of the formula I are employed here in which R² is a methyl or ethyl group.

Furthermore, the use of the compounds of the formula I is preferred in which R¹ and R² independently of one another are methyl or ethyl, X is a hydrogen atom or hydroxyl group and n is an integer from 3 to 5.

The use of 1-(5-hydroxy-5-methylhexyl)-3-methylxanthine is very particularly preferred.

The invention furthermore relates to novel compounds of the formula I,

and/or a physiologically tolerable salt of the compound of the formula I,

and/or a stereoisomeric form of the compound of the formula 1, where

R¹ is methyl- or ethyl,

R² is alkyl having 1 to 4 carbon atoms,

X is a hydrogen atom or hydroxyl group and

n is an integer from 1 to 5,

where 1-(5-hydroxy-5-methylhexyl)-3-methylxanthine is excluded.

Compounds of the formula I are preferred here in which R² is methyl or ethyl, where R¹ and R² are not simultaneously methyl if X is a hydrogen atom and n is the number 4.

Furthermore, the compounds of the formula I are also preferred in which X is a hydrogen atom, where R¹ and R² are not simultaneously methyl if n is the number 4.

Particularly preferred compounds of the formula I are finally those in which

R¹ is methyl,

R² is methyl or ethyl,

X is a hydrogen atom and

n is an integer from 1 to 5,

where R² is not methyl if n is the number 4.

The invention furthermore relates to an analogous process for the preparation of the novel compounds of the formula I whose embodiments are described in principle in WO 87/00523. A procedure is then advantageously used in which a 3,7-disubstituted xanthine derivative of the formula II

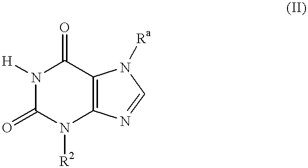

in which R² is an alkyl group having 1 to 4 carbon atoms and R^{a} is a readily eliminable leaving group, for example the hydrolytically removable meth-, eth-, prop- or butoxymethyl radical or the reductively removable benzyl or diphenylmethyl group having unsubstituted or substituted phenyl rings, is expediently reacted in the presence of a basic condensing agent or in the form of its salts

a) with an alkylating agent of the formula III

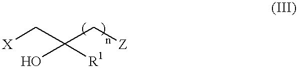

in which R¹, X and n have the abovementioned meanings and Z is halogen, preferably chlorine, bromine or iodine, or a sulfonic acid ester or phosphoric acid ester group,

to give a 1,3,7-trisubstituted xanthine of the formula IV

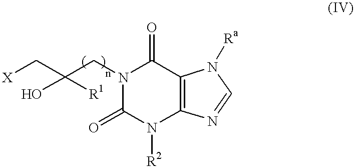

where R¹, R², R^{a}, X and n have the meanings defined above,

or alternatively in the case where X is hydrogen,

b) with a keto compound of the formula V

H₃C—CO—(CH₂)ₙ—Z (V)

in which n and Z have the abovementioned meanings, to give a 1,3,7-trisubstituted xanthine of the formula VI

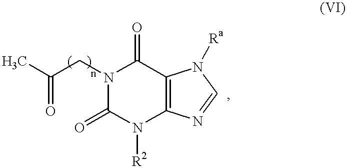

this is then converted using a methyl- or ethyl-metal compound (R¹—M), preferably methyl- or ethyllithium (R¹—Li) or the corresponding Grignard compounds (R¹—MgHal), with reductive alkylation of the carbonyl group into a 1,3,7-trisubstituted xanthine of the formula VII

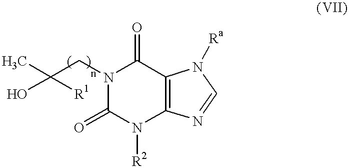

in which R¹, R², R^{a} and n have the abovementioned meanings,

or alternatively in the case where X is hydrogen and R¹ is methyl,

c) with a carboxylic acid ester of the formula VIII

(C₁-C₄)alkyl-O-CO-(cH₂)ₙ—z (VIII)

in which n and Z have the abovementioned meanings, to give a 1,3,7-trisubstituted xanthine of the formula IX

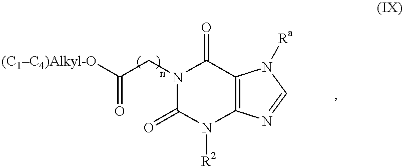

this is then converted with two equivalents of a methyl-metal compound, preferably CH₃—Li or CH₃—MgHal, with double reductive alkylation of the ester function into a 1,3,7-trisubstituted xanthine of the formula X

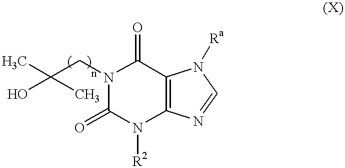

where R², R^{a} and n have the abovementioned meanings,

and finally the xanthine of the formula I according to the invention is obtained by elimination the leaving group R^{a} from the intermediate of the formula IV, VII or X.

The 3,7-disubstituted xanthines of the formula II used in this context as starting materials and alkylating agents of the formula III, V and VIII are for the most part known or can easily be prepared by methods known from the literature (see, for example, WO 87/00523). Thus the tertiary alcohols of the formula III can be obtained, for example, by organometallic synthesis by reacting the sterically unhindered haloketones of the formula Hal-(CH₂)ₙ—CO—Co₂X in a so-called synthesis reaction with reductive alkylation of the carbonyl group using alkyl metal compounds R¹—M, in which M is metal, especially magnesium, zinc or lithium, for example in the form of the alkylmagnesium halides R¹—MgHal (Grignard compounds) or the alkyllithium compounds R¹—Li, under customary conditions. A similar reaction of the haloketones of formula Hal—(CH₂)ₙ—CO—R¹ with methylmagnesium halides or methyllithium likewise leads to compounds of the formula III in which X is hydrogen. A convenient access to compounds of the formula III in which R¹ is methyl and X is a hydrogen atom is also offered by the reaction of alkyl w-haloalkanoates (Hal—(CH₂)ₙ-COO-alkyl) with two equivalents of a methyl-metal compound, the ester reacting via the ketone to give the tertiary alcohol with introduction of two methyl radicals. In the same manner, w-hydroxycarboxylic acid esters can be converted into diols using methyl-metal compounds, without or with protection of the hydroxyl group, for example in the form of the tetrahydropyran-2-yl or methoxymethyl ether or, if appropriate, also the lactones as cyclic esters, from which active alkylating agents of the formula III are obtained by selective esterification of the primary hydroxyl function with sulfonic acid or phosphoric acid halides or anhydrides.

The reaction of the disubstituted xanthine derivatives of the formula II with the alkylating agents of the formula III, V or VIII concerned is usually carried out in a dispersing agent or solvent which is inert to the reaction participants. Possible solvents are especially dipolar, aprotic solvents, for example formamide, di-methylformamide, dimethylacetamide, N-methylpyrrolidone, tetramethylurea, hexamethylphosphoric triamide, dimethyl sulfoxide, acetone or butanone; however, alcohols, such as methanol, ethylene glycol and its mono- or di(C₁-C₄)-alkyl ethers, ethanol, propanol, isopropanol and the various butanols; hydrocarbons, such as benzene, toluene or xylenes; halogenated hydrocarbons, such as dichloromethane or chloroform; pyridine and also mixtures of the solvents mentioned or mixtures thereof with water can also be used.

The alkylation reactions are expediently carried out in the presence of a basic condensing agent. Those suitable for this are, for example, alkali metal or alkaline earth metal hydroxides, carbonates, hydrides or alkoxides and organic bases, such as trialkylamines, e.g. triethyl- or tributylamine, quaternary ammonium or phosphonium hydroxides and crosslinked resins having fixed, optionally substituted ammonium or phosphonium groups. The xanthine derivatives, however, can also be employed directly in the form of their separately prepared salts, for example the alkali metal, alkaline earth metal or optionally substituted ammonium or phosphonium salts. Furthermore, the disubstituted xanthine compounds can be conveniently alkylated both in the presence of the abovementioned inorganic condensing agents and in the form of their alkali metal or alkaline earth metal salts with the additional aid of so-called phase-transfer catalysts, for example tertiary amines, quaternary ammonium or phosphonium salts or alternatively crown ethers, preferably in a two-phase system under the conditions of a phase-transfer catalysis. Suitable, mostly commercially available phase-transfer catalysts, are, inter alia, tetra(C₁-c₄)alkyl- and methyltrioctyl ammonium and phosphonium salts, methyl, myristyl, phenyl and benzyl-tri(C₁-C₄)alkyl and cetyltrimethylammonium salts and (C₁-C₁₂)alkyl and benzyltriphenylphosphonium salts, where as a rule those compounds which have the larger and more symmetrically constructed cation prove to be more effective. In the procedures described above, the reaction is in general carried out at a reaction temperature of between 0° C. and the boiling point of the reaction medium used in each case, preferably between 20° and 130° C., if appropriate at elevated or reduced pressure, but usually at atmospheric pressure, it being possible for the reaction time to be from less than one hour up to several hours.

In the case of the organometallic reactions of the xanthines VI and IX functionalized in the radical in position 1, the procedure is in principle carried out in the same manner as described for the preparation of the tertiary alcohols of formula III used as alkylating agents. Thus the reductive alkylation of the ketones VI or of the esters IX can be carried out, for example, using alkyl-potassium, -sodium, -lithium, -magnesium, -zinc, -cadmium, -aluminum and -tin compounds. The recently recommended alkyl-titanium and -zirconium compounds (D. Seebach et al., Angew. Chem. 95 (1983), pp. 12-26) can also be employed. As, however, the alkyl-metal compounds of sodium and potassium are prone to side reactions on account of their high reactivity and those of zinc and cadmium are comparatively sluggish to react, the alkyl-lithium and -magnesium (Grignard) compounds are usually preferred.

The strongly nucleophilic organometallic compounds are very sensitive to hydrolysis and oxidation. Their safe handling therefore requires working in anhydrous medium, if appropriate under a protective gas atmosphere. Customary solvents or dispersing agents are principally those which are also suitable for the preparation of the alkyl-metal compounds. Those come especially ethers having one or more ether oxygen atoms, for example diethyl, dipropyl, dibutyl or diisoamyl ether, 1,2-dimethoxyethane, tetrahydrofuran, dioxane, tetrahydropyran, furan and anisole, and aliphatic or aromatic hydrocarbons, such as petroleum ether, cyclohexane, benzene. Toluene, xylenes, diethylbenzenes and tetrahydronaphthalene in question; however, tertiary amines, such as triethylamine, or dipolar, aprotic solvents, for example hexamethylphosphoric triamide, and also mixtures of the solvents mentioned can also be used with success. In the case of reaction of the carbonyl compounds VI or IX with the Grignard compounds of the formula R¹MgHal, a procedure can also advantageously be used in which the organometallic compound is initially introduced in an ether and the ketone or the ester is added dropwise as a solution in dichloromethane or 1,2-dichloroethane. Often recommended is addition of magnesium bromide, which on account of its participation in the complex-like cyclic transition state is able to increase the nucleophilicity of the organometallic compound.

The purification of ketone or ester and organometallic compound is generally carried out at temperatures between −20° and 100° C. preferably between 0° and 60° or at room temperature without external cooling, the alkyl metal compound customarily being used in a slight excess. The reaction is then usually ended by brief heating under reflux, for which, as a rule, time spans of a few minutes up to a few hours are sufficient. The decomposition of the alkoxide formed is preferably carried out using aqueous ammonium chloride solution or dilute acetic acid.

The leaving group R^{a} is eliminated from the compounds of the formulae IV, VII and X with formation of the xanthines of the formula I according to the invention under standard conditions, which were especially developed in the context of the protective group technique in alkaloid and peptide syntheses and can thus be assumed to be widely known.

The benzyl or diphenylmethyl group which is optionally substituted in the phenyl ring is then preferably reductively removed. Beside chemical reduction, in particular of the benzyl compounds with sodium in liquid ammonia, the elimination of the two abovementioned aralkyl groups by catalytic hydrogenolysis with the aid of a noble metal catalyst is preferably suitable for this purpose, the replacement of molecular hydrogen by ammonium formate as a hydrogen donor often having proven suitable. The reaction medium usually used in this case is a lower alcohol, if appropriate with the addition of formic acid or alternatively ammonia; an aprotic solvent, such as dimethylformamide or in particular glacial acetic acid; but also mixtures thereof with water can be used. Suitable hydrogenation catalysts are especially palladium black and palladium on active carbon or barium sulfate, while other noble metals such as platinum, rhodium and ruthenium, as a result of competing nuclear hydrogenation, often cause side reactions and therefore can only be employed to a limited extent. The hydrogenolysis is expediently carried out at temperatures between 20° C. and 100° C. and under atmospheric pressure or preferably slight overpressure up to approximately 10 bar, as a rule reaction times of a few minutes up to several hours being needed. The 1,3,7-trisubstituted xanthines of the formulae IV, VII and X, which carry an alkoxymethyl group in the position of R^{a}, are O,N-acetals and can accordingly be easily demasked under the customary conditions of acidic hydrolysis. Preferred radicals are, for example, the methoxy-, ethoxy-, propoxy- and butoxymethyl group. The reaction is advantageously carried out with warming in dilute mineral acids, such as hydrochloric or sulfuric acid, if appropriate with addition of glacial acetic acid, dioxane, tetrahydrofuran or a lower alcohol as a solubilizer. Occasionally, perchloric acid or organic acids, such as trifluoroacetic, formic and acetic acid, in association with catalytic amounts of mineral acids are also suitable. In principle, the cleavage of the ether group can also be carried out with the aid of Lewis acids, such as zinc bromide and titanium tetrachloride, in anhydrous medium, preferably in dichloromethane or chloroform. In the case of cleavage in mineral acid solution, the reaction temperature is to be selected such that no noticeable dehydration of the tertiary hydroxy-alkyl group in position 1 occurs; it should therefore as a rule not exceed 60° C.

The compounds of the formula I can be deprotonated in position 7 and therefore form salts and solvates with basic agents. Suitable salts for this purpose are preferably the pharmaceutically acceptable alkali metal and alkaline earth metal salts and the salts and solvates with organic bases, for example ethylenediamine, or the basic amino acids lysine, ornithine and arginine. The invention thus also relates to pharmacologically tolerable salts and/or solvates of the 1,3-dialkylxanthines of formula I.

The tertiary 1-(hydroxyalkyl)-3-alkylxanthines of the formula I have an asymmetric carbon atom if X is hydroxyl or X is hydrogen and R¹ is ethyl. These compounds can thus exist in stereoisomeric forms. The invention therefore relates both to the pure stereoisomeric compounds and to mixtures thereof.

The novel xanthine compounds of the formula I according to the invention are outstandingly suitable on account of their useful pharmacological properties for use as active compounds in pharmaceuticals, in particular in those which make possible effective prophylactic and curative treatment of the disorders due to pathological eosinophilia hyperreactivity, such as those of the atopic type, and thus represent a substantial enrichment of pharmaceutical resources. They can either be administered per se, for example in the form of microcapsules, in mixtures with one another or in combination with suitable excipients.

The invention consequently also relates to pharmaceuticals which contain at least one compound of the formula I as active compound, 1-(5-hydroxy-5-methylhexyl)-3-methylxanthine being excluded.

A further aspect of the present invention, which relates to all compounds coming under the formula I, is the production of pharmaceutical preparations for oral, rectal, topical, parenteral or inhalative administration in disorders with a pathologically raised reactivity of the eosinophilic granulocytes. Suitable solid or liquid pharmaceutical preparation forms are, for example, granules, powders, tablets, coated tablets (micro)-capsules, suppositories, syrups, emulsions, suspensions, lotions, creams, ointments, gels, aerosols, drops or injectable solutions in ampoule form and also preparations having protracted release of active compound, in whose preparation auxiliaries, such as excipients, disintegrants, binders, coating agents, swelling agents, glidants or lubricants, flavorings, sweeteners or solubilizers, are customarily used. Auxiliaries which are often used and which may be mentioned are, for example, magnesium carbonate, titanium dioxide, lactose, mannitol and other sugars, talc, milk protein, gelatin, starch, vitamins, cellulose and its derivatives, animal and vegetable oils, polyethylene glycols and solvents, such as, for example, sterile water, alcohols, glycerol and polyhydric alcohols.

The pharmaceutical preparations are preferably prepared and administered in dose units, each unit containing as active constituent a certain dose of a compound of formula I. In the case of solid dose units, such as tablets, capsules and suppositories, this dose can be up to 1000 mg, but preferably 100 to 600 mg, and in the case of injection solutions in ampoule form up to 300 mg, but preferably 20 to 200 mg.

For the treatment of an adult patient—depending on the efficacy of the compounds of formula I in man—daily doses of 100 to 2000 mg of active compound, preferably 300 to 900 mg, are indicated in the case of oral administration and from 10 to 500 mg, preferably 20 to 200 mg, in the case of intravenous administration.

Under certain circumstances, however, higher or lower daily doses may also be appropriate. The administration of the daily dose can be carried out either by single administration in the form of an individual dose unit or else of several smaller dose units or by multiple administration of subdivided doses at specific intervals.

Finally, the xanthine derivatives of the formula I can also—if necessary—be formulated together with other suitable active compounds, for example antihistamines, anticholinergics, β₂-mimetics, phosphodiesterase, phospholipase A₂ and lipoxygenase inhibitors, PAF and leukotriene antagonists, corticosteroids, chromoglycic acid, nedocromil and also cyclosporin A, during the preparation of the abovementioned pharmaceutical preparation forms.

The structure of all compounds described below and compiled in Table 1 was confirmed by elemental analysis and IR as well as ¹H-NMR spectra.

Preparation Examples

### EXAMPLE 1

### 1-(2-Hydroxy-2-methylpropyl)-3-methylxanthine

a) 1-Chloro-2-hydroxy-2-methylpropane

A solution of 46.3 g (0.5 mol) of 1-chloro-2-propanone in 50 ml of anhydrous diethyl ether was added dropwise with stirring at 0° to 5° C. to 44.9 g (0.6 mol) of methyl magnesium chloride in the form of a 20% strength solution in tetrahydrofuran and 200 ml of dry diethyl ether. The mixture was then stirred first at room temperature for one hour and then with boiling under reflux for a further hour, the tertiary alkoxide formed was decomposed by addition of 50% strength aqueous ammonium chloride solution, the ether phase was separated off and the aqueous phase was extracted by shaking with ether. The combined ethereal extracts were washed successively with aqueous sodium hydrogen sulfite and sodium hydrogen carbonate solution and a little water, dried over sodium sulfate, filtered, concentrated under reduced pressure and the liquid residue was subjected to fractional distillation.

Yield: 31.1 g (57.3% of theory) Boiling point: 125-127° C. C₄H₉ ClO (MW=108.6)

It was also possible to prepare the compound in an analogous manner from methyl or ethyl chloroacetate using twice the molar amount of methyl magnesium chloride in yields of around 60% of theory.

b) 7-Benzyl-1-(2-hydroxy-2-methylpropyl)-3-methylxanthine

The mixture of 25.6 g (0.1 mol) of 7-benzyl-3-methylxanthine, 15.2 g (0.11 mol) of potassium carbonate and 11.9 g (0.11 mol) of the tertiary alcohol from stage a) in 500 ml of dimethylformamide was heated at 110° to 120° C. with stirring for 8 hours, then hot-filtered and evaporated under reduced pressure. The residue was taken up with chloroform, washed first with 1 N sodium hydroxide solution, then with water until neutral and dried, the solvent was distilled off in vacuo and the solid residue was recrystallized from ethyl acetate with addition of petroleum ether.

Yield: 26.6 g (81.0% of theory) Melting point: 115-117° C. C₁₇H₂₀N₄O₃ (MW=328.4)

Analysis: Calculated: C, 62.18%; H, 6.14%; N, 17.06%. Found: C, 62.60%; H, 6.18%; N, 17.00%.

It was also possible to prepare the compound by first reacting 7-benzyl-3-methylxanthine with 1-chloro-2-propanone or methyl or ethyl chloroacetate under the reaction conditions previously described to give 7-benzyl-3-methyl-1-(2-oxopropyl)xanthine or 7-benzyl-1-meth(or eth)oxycarbonylmethyl-3-methylxanthine and then reductively methylating the oxopropyl or alkoxycarbonylmethyl side chain with methylmagnesium chloride in anhydrous diethyl ether analogously to stage a).

c) 1-(2-Hydroxy-2-methylpropyl)-3-methylxanthine

13.1 g (0.04 mol) of 7-benzylxanthine from stage b) were hydrogenated with shaking in 200 ml of glacial acetic acid over 1.5 g of palladium (10%) on active carbon at 60 C and 3.5 bar in the course of 100 hours. After cooling, the mixture was blanketed with nitrogen, the catalyst was filtered off, the filtrate was concentrated under reduced pressure and the solid residue was recrystallized from ethyl acetate.

Yield: 7.8 g (81.8% of theory) Melting point: 215-217° C. C₁₀H₁₄N₄O₃ (MW=238.3)

Analysis: Calculated: C, 50.41%; H, 5.92%; N, 23.52%. Found: C, 50.10%; H, 5.90%; N, 23.40%.

### EXAMPLE 2

### 3-Ethyl-1-(2-hydroxy-2-methylpropyl)xanthine

a) 7-Benzyl-3-ethylxanthine

20 g (0.5 mol) of sodium hydroxide dissolved in 200 ml of water were added to a suspension of 90 g (0.5 mol) of 3-ethylxanthine in 500 ml of methanol and the mixture was stirred at 70° C. for one hour, then treated dropwise at the same temperature with 69.6 g (0.55 mol) of benzyl chloride, and the reaction mixture was held between 70° and 80° C. for 3 hours. It was then cooled, solid was filtered off cold on a suction filter, and the product was washed with water on the suction filter, dissolved hot in 1000 ml of 1 N sodium hydroxide solution, filtered and brought to pH 9.5 slowly with stirring using 4 N hydrochloric acid. The crystallizate was filtered off from the still warm solution, washed with water until chloride-free and dried in vacuo.

Yield: 131 g (96.9% of theory) Melting point: 217-218° C. C₁₄H₁₄N₄O₂ (MW=270.3)

b) 3-Ethyl-1-(2-hydroxy-2-methylpropyl)xanthine

By reaction of 7-benzyl-3-ethylxanthine from stage a) with 1-chloro-2-hydroxy-2-methylpropane from Example 1a) to give 7-benzyl-3-ethyl-1-(2-hydroxy-2-methylpropyl)-xanthine (C₁₈H₂₂N₄O₃ (MW=342.2); yield: 46.1% of theory) analogously to Example 1b) and subsequent hydrogenolytic debenzylation (yield: 97.9% of theory) according to Example 1c), crude final product was obtained which could be purified by recrystallization from ethanol.

Melting point: 217-219° C.

C₁₁H₁₆N₄O₃ (MW=252.3)

Analysis: Calculated: C, 52.37%; H, 6.39%; N 22.21%. Found: C, 52.19%; H, 6.29%; N, 21.75%.

### EXAMPLE 3

### 1-(3-Hydroxy-3-methylbutyl)-3-methylxanthine

a) 1-Chloro-3-hydroxy-3-methylbutane

The compound was prepared from methylmagnesium iodide and 1-chloro-3-butanone (obtainable by addition of hydrogen chloride to methyl vinyl ketone in diethyl ether) or from methylmagnesium chloride and ethyl 3-chloropropionate in dichloromethane as a reaction medium analogously to Example 1a).

Yield: 60-70% of theory Boiling point (18 mbar): 66-68° C. C₅H₁₁ClO (MW=122.6)

b) 7-Benzyl-1-(3-hydroxy-3-methylbutyl)-3-methylxanthine

prepared analogously to Example 1b) from 7-benzyl-3-methylxanthine and the tertiary alcohol from stage a).

Yield: 70% of theory) Melting point: 92-94° C. C₁₈H₂₂N₄O₃ (MW=342.4)

Analysis: Calculated: C, 63.14%; H, 6.48%; N, 16.36%. Found: C, 63.10%; H, 6.43%; N, 16.28%.

c) 1-(3-Hydroxy-3-methylbutyl) -3-methylxanthine

prepared by hydrogenolytic debenzylation of the product from stage b) analogously to Example 1c).

Yield: 87.2% of theory Melting point: 203-205° C. C₁₁H₁₆N₄O₃ (MW=252.3)

Analysis: Calculated: C, 52.37%; H, 6.39%; N, 22.21%. Found: C, 52.13%; H, 6.52%; N, 22.08%.

### EXAMPLE 4

### 3-Ethyl-1-(3-hydroxy-3-methylbutyl)xanthine

a) 7-Benzyl-3-ethyl-1-(3-hydroxy-3-methylbutyl)xanthine prepared analogously to Example 1b from 7-benzyl-3-ethyl-xanthine (Example 2a) and 1-chloro-3-hydroxy-3-methylbutane (Example 3a).

Yield: 71.8% of theory Melting point: 133-135° C. C₁₉H₂₄N₄O₃ (MW=356.4)

b) 3-Ethyl-1-(3-hydroxy-3-methylbutyl)xanthine

obtained according to Example 1c) by hydrogenolytic debenzylation of the product from stage a).

Yield: 88.2% of theory Melting point: 241-243° C. C₁₂H₁₈N₄O₃ (MW=266.3)

Analysis: Calculated: C, 54.12%; H, 6.81%; N, 21.04%. Found: C, 53.89%; H, 6.86%; N, 21.03%.

### EXAMPLE 5

### 1-(4-hydroxy-4-methylpentyl)-3-methylxanthine

a) 7-Benzyl-3-methyl-1-(4-oxopentyl)xanthine

38.4 g (0.15 mol) of 7-benzyl-3-methylxanthine, 22.4 g (0.162 mol) of potassium carbonate and 26.7 g (0.162 mol) of 1-chloro-4-pentanone ethylene ketal in 600 ml of dimethylformamide were first reacted analogously to Example 1b) to give 7-benzyl-1-(4,4-ethylenedioxypentyl)-3-methylxanthine, which was subjected without further purification to a ketal cleavage by heating under reflux for 2 hours in 600 ml of 1 N hydrochloric acid. After neutralization of the mixture using concentrated sodium hydroxide solution, the ketone formed was taken up in chloroform and the chloroform extract was washed with water, dried over sodium sulfate and evaporated to dryness under reduced pressure.

Yield: 50.4 g (98.7% of theory) Melting point: 104-105° C. C₁₈H₂₀N₄O₃ (MW=340.4)

b) 7-Benzyl-1-(4-hydroxy-4-methylpentyl) 3-methylxanthine

A mixture of 9 g (0.12 mol) of methylmagnesium chloride in the form of the commercially available 20% strength solution in tetrahydrofuran and 300 ml of dichloromethane was cooled to −25° C. and then treated dropwise with a solution of 34 g (0.1 mol) of the product from stage a), the temperature climbing to 20° C. The mixture was subsequently stirred at room temperature for a further hour and treated with saturated ammonium chloride solution, the organic phase was separated off, the aqueous phase was extracted several times by shaking with dichloromethane, the combined dichloromethane extract was washed with water, dried and evaporated and the solid residue was recrystallized from ethyl acetate.

Yield: 28.3 g (79.4% of theory) Melting point: 132-133° C. C₁₉H₂₄N₄O₃ (MW=356.4)

c) 1-(4-Hydroxy-4-methylpentyl)-3-methylxanthine

was prepared according to Example 1c) by hydrogenolytic debenzylation of the product from stage b).

Yield: 65.9% of theory Melting point: 188-189° C. C₁₂H₁₈N₄O₃ (MW=266.3)

Analysis: Calculated: C, 54.12%; H, 6.81%; N, 21.04%. Found: C, 53.86%; H, 6.88%; N, 20.93%.

### EXAMPLE 6

### 3-Ethyl-1-(4-hydroxy-4-methylpentyl)xanthine

a) 7-Benzyl-3-ethyl-1-(4-oxopentyl)xanthine

Preparation was carried out analogously to Example 5a) employing 7-benzyl-3-ethylxanthine from Example 2a) as starting substance

Yield: 82.4% of theory Melting point: 139-141° C. C₁₉H₂₂N₄O₃ (MW=354.4)

b) 7-Benzyl-3-ethyl-1-(4-hydroxy-4-methylpentyl)xanthine

The product from stage a) was reacted with methyl-magnesium chloride analogously to Example 5b).

Yield: 81.9% of theory Melting point: 155-157° C. C₂₀H₂₆N₄O₃ (MW=370.5)

Analysis: Calculated: C, 64.84%; H, 7.07%; N, 15.12%. Found: C, 64.95%; H, 7.18%; N, 15.10%.

c) 3-Ethyl-1-(4-hydroxy-4-methylpentyl)xanthine

The compound was obtained by hydrogenolytic debenzylation of the product from stage b) analogously to Example 1c).

Yield: 71.3% of theory) Melting point: 214-216° C. C₁₃H₂₀N₄O₃ (MW=280.3)

Analysis: Calculated: C, 55.70%; H, 7.19%; N, 19.99%. Found: C, 55.50%; H, 7.20%; N, 20.23%.

### EXAMPLE 7

### 1-(5-Hydroxy-5-methylhexyl)-3-methylxanthine

The preparation methods for this compound are described in detail in PCT Application WO 87/00523.

### EXAMPLE 8

### 1-(5,6-Dihydroxy-5-methylhexyl)-3-methylxanthine

a) 1-Chloro-5,6-isopropylidenedioxy-5-methylhexane 1000 ml of anhydrous dimethyl sulfoxide were added dropwise in the course of 10 minutes at 40° C. with stirring to a mixture of 264 g (1.2 mol) of trimethyl-sulfoxonium iodide and 28.8 g (1.2 mol) of sodium hydride which was blanketed with nitrogen. After evolution of gas was complete (about 2 hours), a solution of 134.6 g (1 mol) of 1-chloro-5-hexanone in 30 ml of dimethyl-sulfoxide was added dropwise in the course of approximately 20 minutes. The mixture was subsequently stirred at room temperature for 2 hours and slowly treated with 500 ml of ice-water with ice-cooling and the 1-chloro-5,6-epoxy-5-methylhexane formed was extracted with diethyl ether (yield: 130.5 g (87.8% of theory); C₇H₁₃ClO (MW=148.6)). For hydrolytic cleavage of the epoxide ring, the product was stirred at room temperature for 5 days in a mixture of 60 ml of water, 600 ml of tetrahydrofuran and 1 ml of 70% strength perchloric acid. It was then neutralized with sodium carbonate solution, the tetrahydrofuran was distilled off to the greatest possible extent and the resulting 1-chloro-5,6-dihydroxy-5-methylhexane was extracted with chloroform. (Yield: 124.8 g (85.3% of theory); C₇H₁₅ClO₂ (MW=166.6)).

The diol was then converted into the dioxolane in a conventional manner using 2,2-dimethoxypropane in acetone with acid catalysis.

Yield: 67.2% of theory Boiling point (0.5 mbar): 84-86° C. c₁₀H₁₉ClO₂ (MW=206.7)

b) 1-(5,6-dihydroxy-5-methylhexyl)-3-methylxanthine

The diol from stage a) could be reacted quantitatively with 7-ethoxymethyl-3-methylxanthine analogously to Example 1b) to give 7-ethoxymethyl-1-(5,6-isopropylidenedioxy-5-methylhexyl)-3-methylxanthine (C₁₉H₃₀N₄O₅, MW=394.5), from which, by acidic hydrolysis with simultaneous opening of the dioxolane ring and removal of the ethoxymethyl radical in position 7, the final product was obtained. For this purpose, 19.7 g (0.05 mol) of the xanthine compound were heated at 70° C. with stirring for 15 hours in a mixture of 300 ml of 1 N hydrochloric acid and 30 ml of glacial acetic acid, and, after cooling, the mixture was rendered alkaline with sodium carbonate and washed with chloroform, then neutralized with 1 N hydrochloric acid and extracted with chloroform. After filtration on a silica gel column in the eluent chloroform/methanol (10:1), the evaporation residue was recrystallized from ethyl acetate.

Yield: 11.5 g (77.6% of theory) Melting point: 181-182° C. C₁₃H₂₀N₄O₄ (MW=296.3)

Analysis: Calculated: C, 52.69%; H, 6.80%; N, 18.91%. Found: C, 52.46%; H, 6.90%; N, 18.66%.

### EXAMPLE 9

### 1-(5-Hydroxy-5-methylheptyl)-3-methylxanthine

7-Benzyl-3-methyl-1-(5-oxohexyl)xanthine, prepared from 7-benzyl-3-methylxanthine and 1-chloro-5-hexanone analogously to Example 1b), was reductively ethylated on the keto group with ethylmagnesium chloride according to Example 5b) and the 7-benzyl-1-(5-hydroxy-5-methylheptyl)-3-methylxanthine obtained in this process was then hydrogenolytically debenzylated under the conditions of Example 1c).

Yield: 70.2% of theory Melting point: 169-170° C. C₁₄H₂₂N₄O₃ (MW=294.4)

Analysis: Calculated: C, 57.13%; H, 7.53%; N, 19.03%. Found: C, 56.90%; H, 7.55%; N, 18.96%.

### EXAMPLE 10

### 3-Ethyl-1-(5-hydroxy-5-methylhexyl)xanthine

7-Benzyl-3-ethyl-1- (5-hydroxy-5-methylhexyl)xanthine, prepared from 7-benzyl-3-ethylxanthine (Example 2a) and 1-chloro-5-hydroxy-5-methylhexane (WO 87/00523) according to Example 1b) in a yield of 65% of theory (C₂₁H₂₈N₄O₃; MW=384.5); melting point: 112-114° C.) was hydrogenolytically debenzylated using ammonium formate as a source of hydrogen. To do this, 3.84 g (0.01 mol) of the benzyl compound and 1.0 g (0.016 mol) of ammonium formate in 30 ml of ethanol were stirred over 2 g of palladium (10%) on active carbon at 35° C. for several days, the successive addition of further ammonium formate up to a total amount of 4.4 g (0.07 mol) having proven suitable. The mixture was filtered, the filtrate was concentrated, the residue was taken up in sodium carbonate solution and washed with chloroform, the aqueous phase was brought to pH 4 using hydrochloric acid, the product was extracted by shaking with chloroform and, after drying and evaporating, it was recrystallized from ethyl acetate.

Yield: 2.0 g (67.9% of theory) Melting point: 180-182° C. C₁₄H₂₂N₄O₃ (MW=294.4)

Analysis: Calculated: C, 57.12%; H, 7.53%; N, 19.04%. Found: C, 56.77%; H, 7.66%; N, 18.93%.

### EXAMPLE 11

### 3-Ethyl-1-(5-hydroxy-5-methylheptyl)xanthine

7-Benzyl-3-ethylxanthine (Example 2a) and 1-chloro-5-hexanone were reacted analogously to Example 1b) to give 7-benzyl-3-ethyl-1-(5-oxohexyl)xanthine (C₂₀H₂₄N₄O₃; MW=368.4; yield: 81.7% of theory; melting point: 123-125° C.). Reductive ethylation of the keto group with ethylmagnesium chloride according to Example 5b) yielded 7-benzyl-3-ethyl-1-(5-hydroxy-5-methylheptyl)xanthine (C₂₂H₃₀N₄O₃, MW=398.5; yield: 86.9% of theory; melting point: 93-94° C.), which was hydrogenolytically debenzylated analogously to Example 10. The final product could be recrystallized from ethanol.

Yield: 66.5% of theory Melting point: 165-166° C. C₁₅H₂₄N₄O₃ (MW=308.4)

Analysis: Calculated: C, 58.42%; H, 7.84%; N, 18.17%. Found: C, 58.30%; H, 8.05%; N, 18.33%.

### EXAMPLE 12

### 1-(6-Hydroxy-6-methylheptyl)-3-methylxanthine

7-Benzyl-1-(6-hydroxy-6-methylheptyl)-3-methylxanthine (C₂₁H28N₄O₃, MW=384.5; melting point: 83-85° C.), prepared with a yield of 77.5% from 7-benzyl-3-methylxanthine and 1-bromo-6-hydroxy-6-methylheptane (WO 87/00523) analogously to Example 1b), was hydrogenolytically debenzylated according to Example 1c).

Yield: 82.2% of theory Melting point: 166-167° C. C₁₄H₂₂N₄O₃ (MW=294.4)

Analysis: Calculated: C, 57.12%; H, 7.53%; N, 19.04%. Found: C, 56.82%; H, 7.74%; N, 19.01%.

### EXAMPLE 13

### 3-Ethyl-1-(6-hydroxy-6-methylheptyl)xanthine

The reaction sequence was carried out with 7-benzyl-3-ethylxanthine from Example 2a) according to Example 12, the hydrogenolytic debenzylation being carried out with ammonium formate analogously to Example 10.

Yield: 72.4% of theory Melting point: 163-165° C. C₁₅H₂₄N₄O₃ (MW 308.4)

Analysis: Calculated: C, 58.42%; H, 7.84%; N, 18.17%. Found: C, 57.83%; H, 7.64%; N, 18.04%.

TABLE 1
Compounds of formula I

| | |
|---|---|
| | (I) |
| 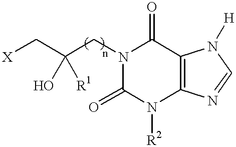 | |

| Example | n | X | R¹ | R² | Melting point ° C. |
|---|---|---|---|---|---|
| 1 | 1 | H | CH₃ | CH₃ | 215-217 |
| 2 | 1 | H | CH₃ | C₂H₅ | 217-219 |
| 3 | 2 | H | CH₃ | CH₃ | 203-205 |
| 4 | 2 | H | CH₃ | C₂H₅ | 241-243 |
| 5 | 3 | H | CH₃ | CH₃ | 188-189 |
| 6 | 3 | H | CH₃ | C₂H₅ | 214-216 |
| 7 | 4 | H | CH₃ | CH₃ | 192-193 |
| 8 | 4 | OH | CH₃ | CH₃ | 181-182 |
| 9 | 4 | H | C₂H₅ | CH₃ | 169-170 |
| 10 | 4 | H | CH₃ | C₂H₅ | 180-182 |
| 11 | 4 | H | C₂H₅ | C₂H₅ | 165-166 |
| 12 | 5 | H | CH₃ | CH₃ | 166-167 |
| 13 | 5 | H | CH₃ | C₂H₅ | 163-165 |

Pharmacological Testing and Results

1. Inhibitory action against the proinflammatory mediators of the early-phase reaction

The inhibitory action of the compounds of formula I on the proinflammatory early-phase mediators histamine, PAF and leukotriene D₄ (LTD₄) was investigated on isolated segments of the respiratory tract organs of albino guinea pigs, the inhibition of the contractions which can be caused by these mediators being used as measurement parameters.

To carry out the experiment, freshly prepared organs of male animals were used in each case. The trachea was divided into its rings, of which 5 tracheal rings in each case were linked with silk thread to give a chain, suspended under a tension of 0.5 g in an organ bath containing Tyrode solution at 37° C. through which was bubbled 95% O_{2/}5% CO₂, and contracted by addition of histamine dihydrochloride (bath concentration: 3×10⁻⁷ g/ml) in the absence (control experiment) or in the presence of the test substances.

The lungs were cut longitudinally into 2 to 3 strips, using which the process as described above was carried out. The tension, however, was 1 g and the contraction was induced by PAF or LTD₄ at a bath concentration of 10⁻⁹ or 10⁻⁸ g/mi.

Each experiment comprised the parallel investigation of 6 organ preparations (n=6).

The assessment of the action of the preparation was carried out with the aid of the IC₅₀ values, which represent that concentration in μg/ml at which the organ contraction produced in the control experiment was reduced by half. The results are compiled in Table 2.

TABLE 2
Inhibitory action on the proinflammatory
early-phase mediators

| | Anticonstrictive action | | |
|---|---|---|---|
| | (IC₅₀ in μg/ml) | | |
| Compound from | Histamine | PAT | LTD₄ |
| Example | trachea | lung | lung |
| 4 | 10 | 1-3 | |
| 5 | 30 | 3 | 10 |
| 6 | 30 | 3 | 1-3 |
| 7 | 10-30 | 10-30 | 10-30 |
| 8 | 10-30 | 6 | 10 |
| 9 | 10-30 | 10 | 10-30 |
| 12 | 3-10 | 3-6 | 6-10 |
| 13 | 10-30 | 1 | |
| Pentoxifylline | 30-60 | 3 | 30-60 |
| Torbafylline | 60 | 3-6 | 10-30 |

2. Inhibition of the antigen-induced early-phase reaction in the presensitized guinea pig

Albino guinea pigs of both sexes having a body weight of 180 to 220 g were sensitized on two successive days by subcutaneous administration of 1 mg of ovalbumin (0.1% strength dissolved in physiological saline solution) in each case.

20 days later, the experiment was carried out according to the method of Konzett and Rössler (Arch. exp. Path. und Pharmak. (1940) 195: 75). For this purpose, the animals were anesthetized with pentobarbital, artificially ventilated, treated with alcuronium chloride to exclude spontaneous respiration and divided into groups of 6 animals in each case. By means of intravenous administration of ovalbumin as an antigen in a dose of 1 mg/kg, a long-lasting asthma attack was induced as a result of an acute bronchospasm induced by mediators in the course of the asthmatic early reaction, the intensity of which was quantified by means of the contraction height in the thoracogram.

The test preparations were likewise administered intravenously 15 minutes before antigen provocation. Instead of this, the animals of the control group received pure 0.9% strength saline solution. To assess the action of the preparation, the number of animals of the respective collective in which the asthma reaction was reduced by at least 40% relative to the control animals was determined. The results are shown in Table 3.

3. Inhibition of eosinophil activation by mediators of the late-phase reaction

The inhibitory action of the xanthines of formula I on the activation ability of human eosinophilic granulocytes by means of the late-phase mediators IL-5, GM-CSF, C5a and PAF was investigated with the aid of the lucigenin-dependent chemiluminescence (CL) reaction.

TABLE 3
Inhibition of the antigen-induced early-phase
reaction in the guinea pig

| Compound | Protected animals after i.v. administration of | | | |
|---|---|---|---|---|
| from | 10 mg/kg | | 25 mg/kg | |
| Example | number | % proportion | number | % proportion |
| 2 | 2/6 | 33 | 4/6 | 67 |
| 4 | 1/6 | 17 | 3/6 | 50 |
| 7 | 2/6 | 33 | 4/5 | 80 |
| 8 | 3/6 | 50 | 3/6 | 50 |
| 9 | 4/6 | 67 | 5/6 | 83 |
| 12 | 3/6 | 50 | 4/6 | 67 |
| Pentoxifylline | 1/6 | 17 | 2/6 | 33 |
| Torbafylline | 0/6 | 0 | 0/6 | 0 |

For this purpose, purified eosinophils were obtained from venous human blood according to known processes (Arch. Dermatol. Res. (1987) 279: 470-477 and J. Invest. Dermatol. (1986) 86: 523-528), pretreated with the test substances at the concentration 100 μM or with pure water (positive control (A)) at 37° C. for 10 minutes and then activated with IL-5 (10² U/ml), GM-CSF (10³ U/ml), C5a (10⁻⁷ M) or PAF (10⁻⁶ M) or treated with water for the determination of the basal activity (B). The CL reaction was monitored over the course of 30 minutes measuring at 37° C. The residual activity of the cells pretreated with the test substances was calculated according to the following formula in percent of that of the positive control:

$$\frac{{CL}_{X} - {CL}_{B}}{{CL}_{A} - {CL}_{B}} \times 100$$

CL_{X} describes the activity after stimulation of the cells pretreated with the xanthines,

CL_{A} describes the activity after stimulation of the cells pretreated with water and

CL_{B} describes the basal activity of unstimulated cells pretreated with water.

The test results are shown in Table 4.

TABLE 4
Inhibition of eosinophil activation by late-
phase mediators

| | |
|---|---|
| | Residual activity in % |
| Compound | of the positive control A |
| from | after stimulation with |

| Example | IL-5 | GM-CSF | C5a | PAF |
|---|---|---|---|---|
| 7 | 42 | 63 | 19 | 17 |
| Pentoxifylline | 57 | 70 | 56 | 45 |
| Torbafylline | 58 | 125 | 51 | 58 |

4. Inhibition of the antigen-induced late-phase reaction

In a chronic long-term experiment on guinea pigs provoked with human serum antigen, the inhibitory action of the compounds of the formula I on the pathologically raised chemotactic infiltration of eosinophilic granulocytes into the peritoneal space (in vivo) and their functional state (ex vivo) was investigated. The animals of the preparation group (n=6) were treated with the test substance at a daily oral dose of 80 mg/kg for 15 weeks, while the animals of the control group (n=6) received the vehicle (carboxymethylcellulose). After the third week of treatment, all 12 animals were provoked by weekly intraperitoneal administration of 1 ml of human serum antigen and 48 hours later in each case subjected to a peritoneal lavage with 50 ml of 5% strength glucose solution in which the number of infiltrated eosinophils and, after their isolation on discontinuous Percoll density gradients (purity >95%; V ability >98%), the reactivity to PAF and C5a were determined in a comparison between the two animal collectives by flow cytometry with the aid of actin polymerization and by means of the Boyden chamber technique.

In this connection, for example, the compound from Preparation Example 7 caused a significant decrease (p<0.01) in the number of eosinophils which migrated into the peritoneal space (34.9 4.8%; ×SD) compared with the control animals (42.2 5.8%). Moreover, the eosinophils of the treated animals, compared with those of the control animals, showed a significant reduction (p<0.05) of the chemotactic migration induced by PAF or C5a; the initial phase (<10s) of actin polymerization induced by PAF (10 nM) was also significantly decreased. This is a clear confirmation of the fact that the compounds of the formula I reduce the antigen-induced, pathological hyperreactivity of eosinophilic granulocytes in the inflammatory tissue and are therefore particularly suitable for the prophylaxis and treatment of disorders of the atopic type.

1. The use of at least one compound of the formula I 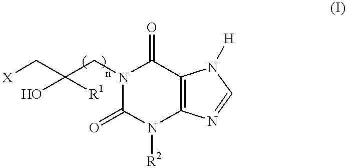
and/or a physiologically tolerable salt of the compound of the formula I and/or a stereoisomeric form of the compound of the formula I, where
R¹ is a methyl or ethyl group,
R² is an alkyl group having I to 4 carbon atoms and
X is a hydrogen atom or a hydroxyl group and
n is an integer from 1 to 5,
for the production of pharmaceuticals for the reduction of the pathological hyperreactivity of eosinophilic granulocytes.

2. The use as claimed in claim 1, wherein at least one compound of the formula I or its salt is employed in which R² is methyl or ethyl.

3. The use as claimed in claim 1 or 2, wherein at least one compound of the formula I or its salt is employed in which
R¹ and R² independently of one another are methyl or ethyl,
X is a hydrogen atom or hydroxyl group and
n is an integer from to 5.

4. The use as claimed in one or more of claims 1 to 3, wherein 1-(5-hydroxy-5-methylhexyl)-3-methylxanthine or its salt is employed.

5. The use as claimed in one or more of claims 1 to 4 for the treatment and/or prophylaxis of disorders of the atopic type.

6. The use as claimed in one or more of claims 1 to 5 for the treatment and/or prophylaxis of anaphylaxis, allergic bronchial asthma, allergic rhinitis and conjunctivitis, allergic urticaria, allergic gastroenteritis and atopic dermatitis.

7. The use as claimed in one or more of claims 1 to 6 for oral, rectal, topical, parenteral or inhalative administration.

8. The use as claimed in claim 7, wherein an effective amount of at least one compound from the antihistamines, anticholinergics, β₂-mimetics, phosphodiesterase, phospholipase A₂ and lipoxygenase inhibitors, PAF and leukotriene antagonists, corticosteroids, chromoglycic acid, nedocromil and cyclosporin A group is additionally employed.

9. A compound of the formula I
and/or a physiologically tolerable salt of the compound of the formula I,
and/or a stereoisomeric form of the compound of the formula I, where
R¹ is methyl- or ethyl,
R² is alkyl having 1 to 4 carbon atoms,
X is a hydrogen atom or hydroxyl group and
n is an integer from 1 to 5,
where the compound of the formula I in which R¹ and R² are simultaneously methyl, X is a hydrogen atom and n is the number 4 is excluded.

10. The compound as claimed in claim 9, wherein in formula I R² is methyl or ethyl.

11. The compound as claimed in claim 9 or 10, wherein in formula I the radical X is a hydrogen atom.

12. The compound as claimed in claims 10 and 11, wherein in formula I
R¹ is methyl,
R² is methyl or ethyl,
X is a hydrogen atom and
n is an integer from 1 to 5.

13. A process for the preparation of the compounds of the formula I as claimed in claims 9 to 12, wherein a 3,7-disubstituted xanthine derivative of the formula II 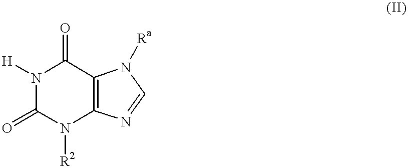
in which R² is an alkyl group having 1 to 4 carbon atoms and R^{a} is a readily eliminable leaving group in the form of the hydrolytically removable meth-, eth-, prop- or butoxymethyl radical or the reductively removable benzyl or diphenylmethyl group having unsubstituted or substituted phenyl rings,
is expediently -reacted in the presence of a basic condensing agent or in the form of its salts
a) with an alkylating agent of the formula III 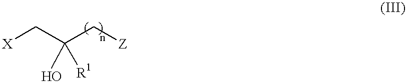
in which R¹, X and n have the abovementioned meanings and Z is chlorine, bromine, iodine or a sulfonic acid ester or phosphoric acid ester group, to give a 1,3,7-trisubstituted xanthine of the formula IV 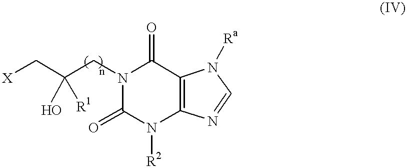
where R¹, R², R^{a}, X and n have the meanings defined above,
or alternatively in the case where X is hydrogen,
b) with a keto compound of the formula V
H₃C—CO—(CH₂)ₙ—Z (V)
in which n and Z have the abovementioned meanings, to give a 1,3,7-trisubstituted xanthine of the formula VI 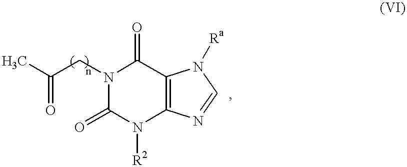
this is then converted using a methyl- or ethyl-metal compound (R¹—M) in the form of methyl- or ethyllithium (R¹—Li) or the corresponding Grignard compounds (R¹—MgHal) with reductive alkylation of the carbonyl group into a 1,3,7-trisubstituted xanthine of the formula VII 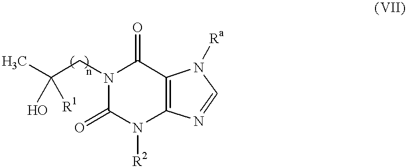
in which R¹, R², R^{a} and n have the abovementioned meanings,
or alternatively in the case where X is a hydrogen atom and R¹ is methyl,
c) with a carboxylic acid ester of the formula VIII
(C₁—C₄)alkyl—O—CO—(cH₂)ₙZ (VIII)
in which n and Z have the abovementioned meanings, 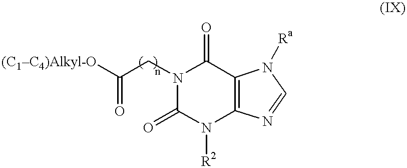
to give a 1,3,7-trisubstituted xanthine of the formula IX, this is then converted with two equivalents of a methyl-metal compound in the form of CH3—Li or CH₃—MgHal with double reductive alkylation of the ester function into a 1,3,7-trisubstituted xanthine of the formula X 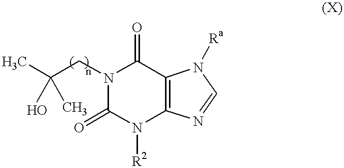
where R², R^{a} and n have the abovementioned meanings,
and finally the leaving group R^{a} is eliminated from the intermediate compound of the formula IV, VII or X with formation of the xanthine of the formula I and this, if desired, is converted into a pharmaceutically acceptable salt.

14. A pharmaceutical, which comprises a therapeutically effective amount of at least one compound of the formula I as claimed in one or more of claims 9 to 12 or prepared as claimed in claim 13.

15. A process for the production of a pharmaceutical as claimed in claim 14, wherein at least one compound of the formula I as claimed in one or more of claims 9 to 12 is brought into a suitable administration form using pharmaceutically suitable and physiologically tolerable excipients and additives, diluents and/or other active compounds or auxiliaries.
